(19) United States
(12) Patent Application Publication
(10) Pub. No.: US 2005/0175716 A1
(43) Pub. Date: Aug. 11, 2005

(54) Method and herbal composition for treatment of diabetes

(76) Inventor: Yuan Y. Lee, Northridge, CA (US)

Correspondence Address: RAYMOND Y. CHAN, 108 N. YNEZ AVE., SUITE 128, MONTEREY PARK, CA, 91754, US

(21) Appl. No.: 10/775,284

(22) Filed: Feb. 9, 2004

(51) Int. Cl. A61K 35/78

(52) U.S. Cl. 424/725

(57) ABSTRACT

The present invention is a method and herbal composition of treatment for diabetes comprising the steps of providing a predetermined herbal composition in a predetermined form, providing an administration plan for a predetermined number of days; and administering the herbal composition according to the administration plan for a predetermined number of days. The herbal composition comprises Toona sinensis and Heracleum lanatum which is natural and does not have any adverse side effects. Toona sinensis is the major active ingredient for treatment of diabetes while Heracleum lanatum is used to provide an optimum body conditions for treatment.

### BACKGROUND OF THE PRESENT INVENTION

1. Field of Invention

The present invention relates to a method and herbal composition for treatment of diabetes, and more particularly to the method of treatment for diabetes wherein a natural herbal composition, Toona sinensis, is one of the active ingredients.

2. Description of Related Arts

Diabetes mellitus is a worldwide problem with increasing number of affected population. Diabetes mellitus, in view of Western medicinal science, is characterized by failure in glucose metabolism which is due to absolute or insufficient insulin production, or relative insensitivity to insulin which is the key hormone for glucogenesis. The trigger of the cause of diabetes is remained unknown and the common treatments include oral intake of medicine and insulin injection to restore the relative sensitivity to the insulin or supply a source of insulin for use. These treatments are for managing and control of the glucose level and ineffective in curing the disease and improving the gravity of the disease.

Other contemporary methods have been developed over time for treating diabetes. For example, exercise is one of the best treatments for diabetes, especially for type II diabetes, for increasing the body conditions and hence decreasing the gravity of diabetes. Even though the mechanism of effect of exercise in treatment of diabetes is not really clear at present, the direct correlation between exercise and blood glucose level are confirmative and widely accepted.

Diet management is another contemporary method for treating diabetes. Since the glucose cycle in our body is affected, the uptake of sugar, ranging from monosaccharide, disaccharide and polysaccharide requires special monitoring. However, glucose is the major energy source of our body, any deficiency or excess of glucose is not good to our body. Thus, careful glucose control is required if the disease is not cured.

Some kinds of natural herb have been used for treating different diseases, together with the rise in Traditional Chinese Medicine and in the growing recognizable use of natural herbs for treatment of disease and improving the body conditions, the effectiveness of the use of natural herbs in treatment in medical science is convincing and promising.

For example, Heracleum lanatum, common name cow parsnip, is one of the natural herbs which is used for treatment in diabetes. In the U.S. Pat. No. 5,741,491, the use of an extract derived from Heracleum lanatum and a specie of Populus is disclosed wherein the direct relation between the administration of the extract and the effect of lowering blood glucose was shown. However, there must still much room for further development or breakthrough in the field of herbs science, especially with the increasing knowledge and understandings of Tradition Chinese medicine and herbs, for treatment of diabetes, partly because existing methods of treatment of diabetes are not really curing the disease and partly because the ever growing number of people are suffered from this disease.

On the other hands, there seems no trial or method for curing or lowering the gravity of the disease of diabetes based on the principles and understandings of diabetes. The present day treatments emphasize on management and control of the disease, which may be too disappointing for those who need to have lifetime management and control of the disease. A good control in every food intake and in every medication intake is somewhat a stress which may further deteriorate the health of the diseased. In fact, any effort or method trying to curing or lowering the gravity of the disease of diabetes is essential and necessary for those diseased.

### SUMMARY OF THE PRESENT INVENTION

A main object of the present invention is to provide a method for treatment of diabetes using a herbal composition for improving the conditions of a body.

Another object of the present invention is to provide a method for treatment of improving the conditions of glucose metabolism by using herbal compositions.

Another object of the present invention is to provide a composition for treatment of diabetes wherein the composition comprises a predetermined combination of natural herbs.

Another object of the present invention is to provide a composition for treatment of diabetes wherein the composition comprises a predetermined combination of natural herbs including Heracleum Lanatum and Toona sinensis so as to improving the conditions for glucose metabolism.

Another object of the present invention is to provide a method and a herbal composition for treatment of diabetes wherein the composition comprises a predetermined combination of natural herbs including Heracleum Lanatum and Toona Sinensis so as to improving the conditions for glucose metabolism.

Another object of the present invention is to provide a method and a herbal composition for treatment of diabetes wherein the composition comprises a predetermined combination of natural herbs including Heracleum Lanatum and Toona Sinensis so as to encourage the restoration of balance of a body which is essential for curing the disease.

Another object of the present invention is to provide a method and a herbal composition for treatment of diabetes wherein Toona sinensis and Heracleum Lanatum having anti-inflammation effect resolve an inflammation condition as well as lower a glucose level.

Accordingly, in order to accomplish the above objects, the present invention is a process of treatment for diabetes comprising the steps of
a) providing a predetermined herbal composition in a predetermined form;
b) providing an administration plan for a predetermined number of days; and
c) administering the herbal composition according to the administration plan for a predetermined number of days.

The herbal composition may be prepared by employing a preparation method comprising the steps of:
a) providing a predetermined quantity of a first herbal composition;
b) providing a predetermined quantity of a second herbal composition wherein the first composition and the second composition has a predetermined ratio by weight;
c) adding the first herbal composition and the second herbal composition to form an essential composition having a predetermined volume;
d) adding a predetermined volume of water to the essential composition to form a pre-heat mixture wherein the volume of water and the volume of the essential composition has a predetermined mixing ratio; and
e) heating the pre-heat mixture under a predetermined temperature for a predetermined heating time to form an after-heat mixture.

In the first preferred embodiment of the present invention, the first and the second composition are Toona Sinensis and Heracleum Lanatum respectively, the mixing ratio is 1:1, the predetermined temperature is between room temperature and boiling point of the pre-heat mixture, and the heating time is about 1.5 to 2 hours.

The herbal composition may be prepared into different forms for oral administration such as tablet, syrup, solution and sachet.

These and other objectives, features, and advantages of the present invention will become apparent from the following detailed description, the accompanying drawings, and the appended claims.

### BRIEF DESCRIPTION OF THE DRAWINGS

FIG. 1 is a preparation method of herbal composition for treating diabetes of the present invention.

FIG. 2 is a process of treatment for diabetes by using herbal composition of the present invention.

FIG. 3 is a table embodying a process of treatment for diabetes by experimentally using herbal composition for an adult having type II diabetes and blood glucose level.

### DETAILED DESCRIPTION OF THE PREFERRED EMBODIMENT

The present invention is a method and herbal composition for treatment of diabetes wherein the herbal composition comprises Toona sinensis.

Toona sinensis belongs to the family Meliaceae is originated from China. Toona sinensis is grey, rough dark color in the bark of trunk and a distribution all over the world now. The whole plant, including seeds, epidermal layer of root, bark, stem and leave, has been used for health supplement or for treatment of disease. It is highly recognized by the Chinese practitioners that the leaves extract of Toona sinensis is capable of effectively lowering the blood glucose level of diabetics, even for those diabetics who have extraordinary high blood glucose level and are not responsive to the conventional medications. Furthermore, it is effective in improving the body conditions against development of long-term or related complications, such as body pain, unstable blood pressure, and numbing of four limbs. Toona sinensis has also been used for treating pimples, common cold, waist pain, and arthritis caused by uric acid. For the treatment of diabetes, the whole plant of the Toona sinensis may be used but the leaf portion is preferred.

Research in analyzing the effect of Toona sinensis on treatment for diabetes showed that leaves of Toona sinensis are capable of lowering the blood glucose level of Alloxan induced diabetes in mice without lowering the blood glucose level of normal mice in control group. It stimulates the secretion of insulin and promotes the performance of the lipid tissue of glucose transferase. In fact, both old aged leaves and new shout are effective in treating diabetes.

Anticancer effect of leaves of Toona sinensis has also been studied. It is capable of inhibiting the growth of cancer cells in lung, rectum, bone, liver and ovary. In American Journal of Chinese Medicine (2002), obvious anticancer effect of leaves of Toona sinensis on animals has been observed.

Toona sinensis has also been used for enhancing the health conditions because of its high nutritional value. Preliminary studies on Toona sinensis showed that it could lower the blood pressure and some documentation stated that it has haemostatic effect. Experimental studies of Toona sinensis solution showed that Toona sinensis has haemostatic effect and that it affects the vascular constriction level of rear limbs of frog. Furthermore, Toona sinensis is capable of releasing pain in animals in oral medication form, anti-inflammation in highly concentrated form, promoting coagulation and anti-coagulation in different concentration of Toona sinensis, antioxidant effect in low dose extract, increasing the lifetime expectancy and increasing the strength of respiratory system in septicaemic mice, improving the function of liver and increasing the activity level of sperm.

Toona sinensis is also suitable to be used as food too. It can be used to prepare juice, tea, or cooking and thus it is very convenience to be used for alternative therapy.

According to the first preferred embodiment of the present invention, the herbal composition further comprises Heracleum lanatum.

Heracleum lanatum is a sturdy herbaceous perennial which is native to North America and is found from Labrador to Alaska, south to the mountains of Georgia and west to California. Its common name is cow parsnip. It is one of the important forage for the animals in these areas.

Native Americans used it in a charm and used the cooked roots for intestinal disorders, the seeds in headache remedies, raw pieces of the root to alleviate toothaches when used as stuffing in dental cavities and many other remedies.

In Alaska, the insides of the raw stems and roots are used for sugar extraction. In Arizona, young leaves and stems are used for cooking and roots are used for treating epilepsy. The root is believed to be beneficial in relieving gas and cramps. The hollow basal portion may be dried and cut into small pieces, and used as a salt substitute. The leaves may also be dried and burned, and used as a salt substitute.

It is known that cow parsnip is a remedy for the stomach and nervous system. The root is usually made into a tea for nausea that is persistent nature but does not progress to vomiting, as well as for acid indigestion or heartburn. The seed tincture is effective in settlement of the unsettled stomach and is a good temporary analgesic for sore tooth with less irritating effect to the gums. The root or seeds is antispasmodic to the intestinal tract. However, since cow parsnip contains sterols and saponins, it is not advised to use during pregnancy.

Since diabetes is also a state of inflammation, a lowering of seriousness of inflammation may greatly promote a body condition which in turn provide a maximum body condition to response positively to any treatment of diabetes. Cow parsnip, having an anti-inflammatory effect, is ideal for providing a maximum body condition for treating diabetes.

Cow parsnip may be prepared into a solution by decoction, or into a poultice by heating without boiling for use, or other methods. For example, the root may be decocted and used for ulcerated sore throat, the root and flowers may be pounded together and made into a poultice for external application to the boiled area.

A combination of use of the Toona sinensis and the cow parsnip has been studied in the present invention and it is found that a herbal composition comprising Toona sinensis and cow parsnip is capable of having positive effect on the treatment of diabetes, arthritis and neuralgia and is effective in lowering blood pressure. The cow parsnip posing anti-inflammatory effect on a body, helps to release the tension caused by inflammation, while at the same time, the Toona sinensis, exerts its effect of lowering blood glucose level. Thus, the combination of use of the Toona sinensis and the cow parsnip is interactively providing an elevated effect of treatment diabetes.

According to the preferred embodiment, the present invention is a herbal composition acted as a alternative remedy for promoting health, especially for treating diabetes, arthritis and neuralgia. The herbal composition comprises a predetermined quantity of Toona sinensis and a predetermined quantity of cow parsnip, wherein the Toona sinensis and the cow parsnip has a predetermined composition ratio, T:C, by weight.

When the composition ratio of Toona sinensis and cow parsnip is 1:1, that is to say when the Toona sinensis and the cow parsnip are used in equal weight, the herbal composition is effective in treating diabetes and inflammation. The Toona sinensis is the active ingredient for treating diabetes while the cow parsnip is the active ingredient for providing an optimum environment and conditions for the Toona sinensis. The cow parsnip is effective in treating diabetes related inflammation. Since the cow parsnip is effective in treating inflammation, it can reduce the severity of the inflammatory level of digestive tract including pancreas such that a more anti-inflammatory status of the digestive tract is achieved. On the other hand, the Toona sinensis is capable of having a better environment and thus has its optimum effect in stimulating the secretion of insulin and hence lowering the glucose level. For more severe case of diabetes, the composition ratio is 7:3, which sides on lowering the glucose level but maintains a minimum anti-inflammatory effect for providing an optimum combination for treatment.

On the other hand, when the composition ratio is 3:7, the herbal composition will then be effective in treatment of neuralgia and arthritis.

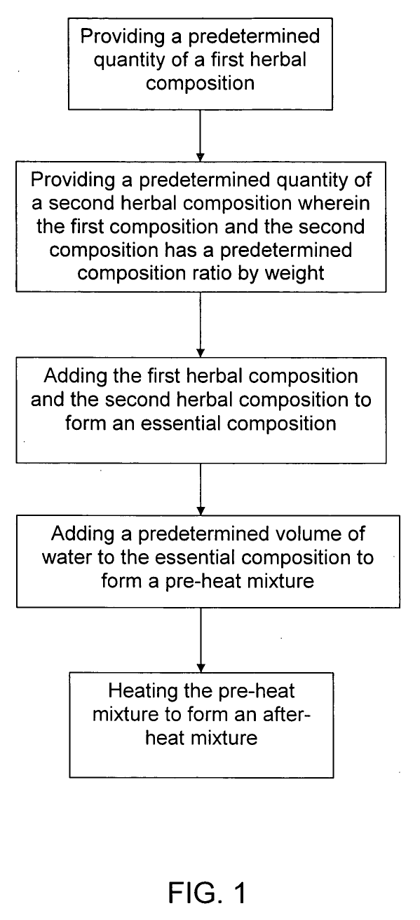

Referring to FIG. 1 of the drawings, the herbal composition is prepared by employing a preparation method comprising the steps of:
a) providing a predetermined quantity of a first herbal composition;
b) providing a predetermined quantity of a second herbal composition wherein the first composition and the second composition has a predetermined composition ratio by weight;
c) adding the first herbal composition and the second herbal composition to form an essential composition having a predetermined volume;
d) adding a predetermined volume of water to the essential composition to form a pre-heat mixture wherein the volume of water and the volume of the essential composition has a predetermined mixing ratio; and
e) heating the pre-heat mixture under a predetermined temperature for a predetermined heating time to form an after-heat mixture.

According to the preferred embodiment of the present invention, the first and the second composition are Toona Sinensis and Heracleum Lanatum respectively, the mixing ratio is 1:1, the predetermined temperature is between room temperature and boiling point of the pre-heat mixture, and the heating time is about 1.5 to 2 hours.

The composition ratio is larger than one, which means the amount of the Toona sinensis used is at least equal to or greater than the amount of Heracleum lanatum, when the herbal composition is used for treating diabetes and the after-heat mixture may then be decanted or filtered to obtain a solution which is suitable for drinking.

A standard single dosage for a normal adult is prepared by using six cup of water. It is suggested that a dosage of at least twice a day, one in the morning and one at night, is preferred for obtaining the optimum effect.

The composition ratio is less than one, which means the amount of the Toona sinensis used is less than the amount of the Heracleum lanatum, when the herbal composition is used for treating neuralgia and arthritis. The after-heat mixture in the preparation method as described may then be decanted or filtered to obtain a solution which is suitable for external application to the infected or inflammatory area. Alternatively, the pre-heat mixture in step (d) may be used directly without heating for external application.

Furthermore, the after-heat mixture of the preparation method of the herbal composition of the present invention may be manufactured into different administrable forms, including tablet, syrup, solution, powder, and sachet forms.

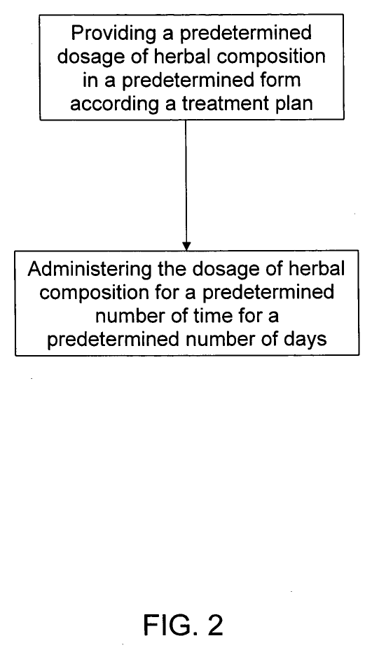

Referring to FIG. 2 of the drawings, the present invention is a process of treatment for diabetes comprising the steps of:
a) preparing a predetermined dosage of herbal composition in a predetermined form according to a treatment plan; and
b) administering the dosage of herbal composition for a predetermined number of time for a predetermined number of days.

According to the preferred embodiment of the present invention, the predetermined dosage of herbal composition is prepared by the preparation method as shown in FIG. 1 of the drawings, wherein the herbal composition comprises the Toona sinensis and the Heracleum lanatum, the predetermined number of time is two times a day, and the predetermined number of days is 30 days, whereby after having the herbal composition for the predetermined number of days, the blood glucose level will be lowered and that the glycohemoglobin A1C level should be improved substantially.

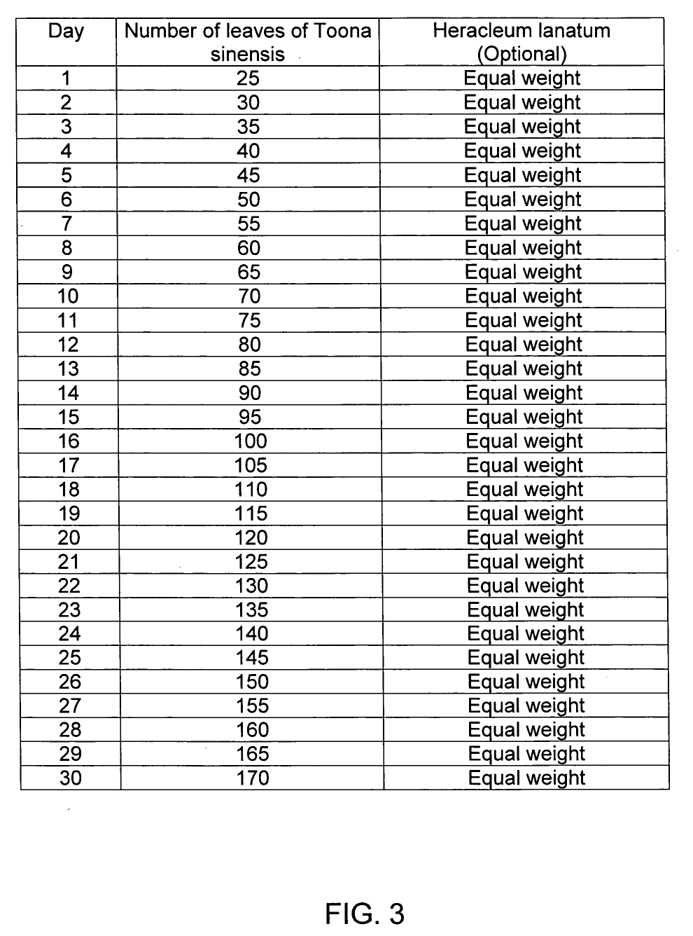

Referring to FIG. 3 of the drawings, a process of treatment for diabetes by using herbal composition for an adult is illustrated.

The process of treatment comprises an administration period of 30 days, from day 1 to day 30 by using herbal compositions. A single dosage of the herbal composition for an adult is prepared by the steps of:
a) providing a predetermined quantity of a first herbal composition;
b) adding a predetermined volume of water to the first herbal composition to form a pre-heat mixture;
c) heating under a predetermined temperature for a predetermined heating time to form an after-heat mixture; and
d) obtaining a final solution from the after-heat mixture.

The first composition is Toona sinensis and the heating temperature is between room temperature and boiling point of the pre-heat mixture, and the heating time is about 1.5 to 2 hours such that a volume of the final solution is reduced into one third of said water in step (b) in volume. The volume of water in step (b) is capable of immersing said first composition in step (a).

In day one, 25 leaves of Toona sinensis is used and 5 leaves of Toona sinensis is added for every following consecutive day. In day 30, 170 leaves of Toona sinensis is used.

Alternatively, a step a′) adding a predetermined amount of a second herbal composition, may be added after step (a) wherein the second herbal composition is cow parsnip in a fresh or raw form. The cow parsnip used may also be in powder form. The amount of the first and the second composition are the same in weight.

For example, in day 1, 25 leaves of Toona sinensis is used as the first herbal composition. Thus, the single dosage of the herbal composition is prepared by the steps of:
a) providing 25 leaves of Toona sinensis;
b) adding six cups of water to form a pre-heat mixture such that said 25 leaves of Toona sinensis is immersed in said water;
c) heating to form an after heat mixture; and
d) obtaining a final solution from the after heat mixture, wherein said final solution is two cups is volume.

Alternatively, when cow parsnip is used optionally, which is in equal weight of the leaves of Toona sinensis, the single dosage of the herbal composition is prepared by the steps of
a) providing 25 leaves of Toona sinensis and a predetermined weight of cow parsnip having a weight equal to a weight of said 25 leaves of Toona sinensis;
b) adding six cups of water to form a pre-heat mixture; and
c) heating to form an after heat mixture having a predetermined amounted to two cups.

During the process, a vegetarian diet and exercise are recommended for improving the body conditions of the adult for having better response to the herbal composition. Two dosages are required for a day, preferably one in the morning and one at night.

A typical example is shown to illustrate the results of the method and herbal composition for treatment of diabetes of the present invention. A living object, male, year of birth 1948, weight 135 pounds and height 5′6″, was suffered from diabetes, and his health conditions before and after the administration of the Toona sinensis and the cow parsnip are recorded.

The initial health condition was recorded on Jan. 3, 2002. The treatment then started and his health condition was recorded on Sep. 11, 2003 and Nov. 10, 2003 respectively. It is shown that the glycohemoglobin A_{1C} (GHb A_{1C}) level decreases from 6.85 to 6.0, representing an improvement of body condition which falls within the normal range. Furthermore, no side or adverse effects have been observed.

The glycohemoglobin A_{1C} is a blood test which is used to measure the changes occurred in hemoglobin. The hemoglobin A_{1c} measurement demonstrates the blood glucose level of the previous few weeks. The reference normal level for GHb A1C is less than 7%.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

1. A herbal composition for treatment of diabetes mellitus comprising compositions of a predetermined quantity of Toona sinensis and a predetermined quantity of Heracleum lanatum.

2. The herbal composition, as recited in claim 1, wherein said Toona sinensis is leaves of Toona sinensis and said Heracleum is roots of Heracleum lanatum.

3. The herbal composition, as recited in claim 1, wherein said quantity of Toona sinensis is not less than said quantity of Heracleum lanatum by weight.

4. The herbal composition, as recited in claim 1, wherein said quantity of Toona sinensis is smaller than said quantity of Heracleum lanatum by weight.

5. The herbal composition, as recited in claim 3, is prepared by a preparation method comprising the steps of:
a) providing said predetermined quantity of Toona sinensis;
b) providing said predetermined quantity of Heracleum lanatum wherein said Toona sinensis and said Heracleum lanatum has a predetermined composition ratio by weight;
c) adding said Toona sinensis and said Heracleum lanatum to form an essential composition having a predetermined volume;
d) adding a predetermined volume of water to said essential composition to form a pre-heat mixture wherein said volume of water and said volume of said essential composition has a predetermined mixing ratio of 1:1; and
e) heating said pre-heat mixture under a predetermined temperature for a predetermined heating time to form an after-heat mixture.

6. The herbal composition, as recited in claim 5, wherein said preparation process further comprises a step f) obtaining a final solution of said herbal composition by decantation.

7. The herbal composition, as recited in claim 4, is prepared by a preparation method comprising the steps of:
a) providing said predetermined quantity of Toona sinensis;
b) providing said predetermined quantity of Heracleum lanatum wherein said Toona sinensis and said Heracleum lanatum has a predetermined composition ratio by weight;
c) adding said Toona sinensis and said Heracleum lanatum to form an essential composition having a predetermined volume;
d) adding a predetermined volume of water to said essential composition to form a pre-heat mixture wherein said volume of water and said volume of said essential composition has a predetermined mixing ratio of 1:1;
e) heating said pre-heat mixture under a predetermined temperature for a predetermined heating time to form an after-heat mixture; and
f) obtaining a final solution of said herbal composition by decantation for external application.

8. A process of treatment for diabetes comprising the steps of:
a) providing a treatment plan having a predetermined number of days and a predetermined number of dosages of herbal composition with respect to each of said number of days;
b) administering said number of dosages of herbal composition with respect to said number of days according to said treatment plan;
wherein said herbal composition comprises a predetermined quantity of Toona sinensis.

9. The process, as recited in claim 8, wherein said predetermined number of days is 30 days from a first day to a thirtieth day.

10. The process, as recited in claim 9, wherein said predetermined number of dosage is 60 and said treatment plan comprises two dosages of herbal composition in each said number of day, a first time in the morning and a second time at night.

11. The process, as recited in claim 8, wherein said Toona sinensis consists of leaves of Toona sinensis.

12. The process, as recited in claim 9, wherein said Toona sinensis consists of leaves of Toona sinensis.

13. The process, as recited in claim 10, wherein said Toona sinensis consists of leaves Toona sinensis.

14. The process, as recited in claim 11, wherein said quantity of Toona sinensis is 25 leaves in said first day and an addition of 5 more leaves in each following consecutive day.

15. The process, as recited in claim 12, wherein said quantity of Toona sinensis is 25 leaves in said first day and an addition of 5 more leaves in each following consecutive day.

16. The process, as recited in claim 13, wherein said quantity of Toona sinensis is 25 leaves in said first day and an addition of 5 more leaves in each following consecutive day.

17. The process, as recited in claim 8, wherein said treatment plan further comprises a predetermined quantity of Heracleum lanatum in said herbal composition, wherein said quantity of Heracleum lanatum and said quantity of Toona sinensis are equal in weight.

18. The process, as recited in claim 9, wherein said treatment plan further comprises a predetermined quantity of Heracleum lanatum in said herbal composition, wherein said quantity of Heracleum lanatum and said quantity of Toona sinensis are equal in weight.

19. The process, as recited in claim 10, wherein said treatment plan further comprises a predetermined quantity of Heracleum lanatum in said herbal composition, wherein said quantity of Heracleum lanatum and said quantity of Toona sinensis are equal in weight.

20. The process, as recited in claim 11, wherein said treatment plan further comprises a predetermined quantity of Heracleum lanatum in said herbal composition, wherein said quantity of Heracleum lanatum and said quantity of Toona sinensis are equal in weight.

21. The process, as recited in claim 12, wherein said treatment plan further comprises a predetermined quantity of Heracleum lanatum in said herbal composition, wherein said quantity of Heracleum lanatum and said quantity of Toona sinensis are equal in weight.

22. The process, as recited in claim 13, wherein said treatment plan further comprises a predetermined quantity of Heracleum lanatum in said herbal composition, wherein said quantity of Heracleum lanatum and said quantity of Toona sinensis are equal in weight.

23. The process, as recited in claim 14, wherein said treatment plan further comprises a predetermined quantity of Heracleum lanatum in said herbal composition, wherein said quantity of Heracleum lanatum and said quantity of Toona sinensis are equal in weight.

24. The process, as recited in claim 15, wherein said treatment plan further comprises a predetermined quantity of Heracleum lanatum in said herbal composition, wherein said quantity of Heracleum lanatum and said quantity of Toona sinensis are equal in weight.

25. The process, as recited in claim 16, wherein said treatment plan further comprises a predetermined quantity of Heracleum lanatum in said herbal composition, wherein said quantity of Heracleum lanatum and said quantity of Toona sinensis are equal in weight.

26. The process, as recited in claim 14, wherein said herbal composition is prepared by the steps of:
a) providing said Toona sinensis having a predetermined volume;
b) adding a predetermined volume of water to said Toona sinensis such that said volume of Toona sinensis and said volume of water are equal;
c) heating said Toona sinensis and water and obtaining a final solution.

27. The process, as recited in claim 16, wherein said herbal composition is prepared by the steps of:
a) providing said Toona sinensis having a predetermined volume;
b) adding a predetermined volume of water to said Toona sinensis such that said volume of Toona sinensis and said volume of water is equal; and
c) heating said Toona sinensis and water and obtaining a final solution.

28. The process, as recited in claim 26, wherein said Toona sinensis is fully immersed in water in step (b).

29. The process, as recited in claim 27, wherein said Toona sinensis is fully immersed in water in step (b).

30. The process, as recited in claim 26, wherein said volume of water in step (b) is six cups and .said final solution in step (c) has a volume of 2 cups.

31. The process, as recited in claim 27, wherein said volume of water in step (b) is six cups and .said final solution in step (c) has a volume of 2 cups.

32. The process, as recited in claim 26, wherein said volume of water in step (b) and said final solution in step (c) has a ratio by volume of 3:1.

33. The process, as recited in claim 27, wherein said volume of water in step (b) and said final solution in step (c) has a ratio by volume of 3:1.

34. The process, as recited in claim 28, wherein said volume of water in step (b) and said final solution in step (c) has a ratio by volume of 3:1.

35. The process, as recited in claim 29, wherein said volume of water in step (b) and said final solution in step (c) has a ratio by volume of 3:1.
